(19) United States
(12) Patent Application Publication
(10) Pub. No.: US 2021/0022906 A1
(43) Pub. Date: Jan. 28, 2021

(54) COMPRESSION APPARATUS FOR PREVENTING SELF-HARM

(71) Applicants: Sungyong PARK, Busan (KR); Kyunghyun NOH, Busan (KR)

(72) Inventors: Sungyong PARK, Busan (KR); Kyunghyun NOH, Busan (KR)

(21) Appl. No.: 16/980,662

(22) PCT Filed: Mar. 14, 2019
(86) PCT No.: PCT/KR2019/002989

§ 371 Date: Sep. 14, 2020

(30) Foreign Application Priority Data
Mar. 14, 2018 (KR) 10-2018-029844

(51) Int. Cl. A61F 5/37; E05B 75/00

(52) U.S. Cl. CPC A61F 5/3761; E05B 75/00; A61F 5/3776

(57) ABSTRACT

A pressure instruments for preventing self-harm includes: a belt unit shaped like a long strap and includes an inner contact surface to be in contact with and wound around a wrist of a patient; a coupler including a first coupler and a second coupler which are attached in sequence from a first end region of the belt unit to a predetermined length section of an outer surface and capable of being stick together; a switch ring coupled to a second end region of the belt unit and allowing the first end region of the belt unit to pass therethrough and be bent reversely so that the first coupler and the second coupler can stick together; and a fastener including a first fastener coupled to an outer surface of the second end region and a second fastener coupled to a region of the coupler and engaged with and locked to the first fastener.

### CROSS REFERENCE TO RELATED APPLICATIONS

This application is a National Stage of Application No. PCT/KR2019/002989 filed Mar. 14, 2019, claiming priority based on Korean Patent Application No. 10-2018-0029844 filed Mar. 14, 2018.

### TECHNICAL FIELD

The disclosure relates to a pressure instruments for preventing self-harm, by which use of a patient's hand is restricted.

### BACKGROUND ART

To prevent a dementia patient and the like person, who have no normal thinking skills, from harming themselves or leaving a restricted place without permission, a pressure instruments for tying up his/her hand has been used. Such a pressure instruments is classified into a glove-type having a structure for accommodating a patient' hand, a wristband type having a structure for compressing a wrist, etc. The wristband-type pressure instruments has a structure like a wristband that is generally fastened and released by Velcro.

However, Velcro has problems that it may lose stickiness with long use and be untied when rubbed against a surrounding thing by a patient.

### DISCLOSURE

### Technical Problem

Accordingly, an aspect of the disclosure is to provide a pressure instruments which is prevented from being untied by a patient and keeps durable tying capability.

### Technical Solution

According to an embodiment of the disclosure, a pressure instruments for preventing self-harm includes: a belt unit shaped like a long strap and includes an inner contact surface to be in contact with and wound around a wrist of a patient; a coupler including a first coupler and a second coupler which are attached in sequence from a first end region of the belt unit to a predetermined length section of an outer surface and capable of being stick together; a switch ring coupled to a second end region of the belt unit and allowing the first end region of the belt unit to pass therethrough and be bent reversely so that the first coupler and the second coupler can stick together; and a fastener including a first fastener coupled to an outer surface of the second end region and a second fastener coupled to a region of the coupler and engaged with and locked to the first fastener. Thus, a dual lock is achieved by the coupler and the fastener.

Here, the fastener may include a pair of locking units pressed and released at lateral sides with respect to a widthwise direction of the belt unit, thereby being prevented from being released even though the fastener is pressed against a wall or other objects.

Further, the fastener may be fastened to the belt unit by a fastening band, and at least one of the fastening bands may be elastically stretchable to thereby fit wrists different in thickness according to patients.

Here, the pressure instruments may further include: a finger accommodator to accommodate fingers of the patient; and an upper connector and a lower connector to connect the finger accommodator and the belt unit along an upper portion and a lower portion of a hand of the patient, respectively, so that the use of the fingers can be restrained, thereby stably maintaining the pressure instruments and improving ventilation.

Further, the pressure instruments may further include a holding belt connected to the belt unit and fastened and tied to a stationary structure, and the holding belt may include: a holding belt main body shaped like a long strap; a holding belt adjustment ring to adjust a length of the holding belt main body; and an end holder supported in the holding belt main body and fastened and tied to the belt unit and the stationary structure, thereby preventing a patient from harming himself/herself because it is possible to tie the pressure instruments to a stationary structure.

Here, the holding belt main body may include rubber projections throughout a predetermined section of an outer surface, so that the end holder can be firmly held.

Further, the pressure instruments may further include a release sensor provided at least one of between the first coupler and the second coupler and between the first fastener and the second fastener and detecting release, so that a manager can be informed that the pressure instruments is untied, thereby preventing a patient from harming himself/herself.

### Advantageous Effects

As described above, according to the disclosure, a patient is prevented from voluntarily releasing a locked state and tying capability is durably kept to securely restrain a patient's hand, thereby having an improved effect on preventing self-harm.

Further, the pressure instruments is stably maintained by stopping a patient from using fingers, and is clean and comfortable by improving ventilation.

Further, a manager is informed that a patient is untied, thereby having a further improved effect on preventing the patient from harming himself/herself.

### DESCRIPTION OF DRAWINGS

FIG. 1 illustrates an example that a pressure instruments according an embodiment of the disclosure is worn on a wrist of a patient.

FIG. 2 illustrates that a pressure instruments is opened.

FIG. 3 illustrates a padding portion according to another embodiment of the disclosure.

FIG. 4 illustrates a holding belt according to another embodiment of the disclosure.

FIG. 5 is a perspective view of an upper connector in a pressure instruments.

FIG. 6 is a perspective view of a lower connector in a pressure instruments.

FIGS. 7 and 8 illustrate an alternative example of identifying whether a pressure instruments is released.

### BEST MODE

Below, a pressure instruments 1 according to embodiments of the disclosure will be described in detail with reference to the accompanying drawings.

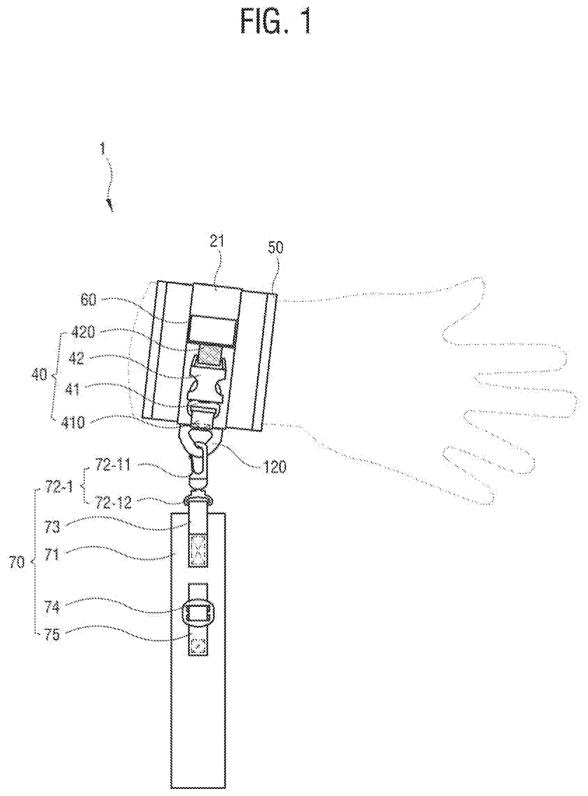

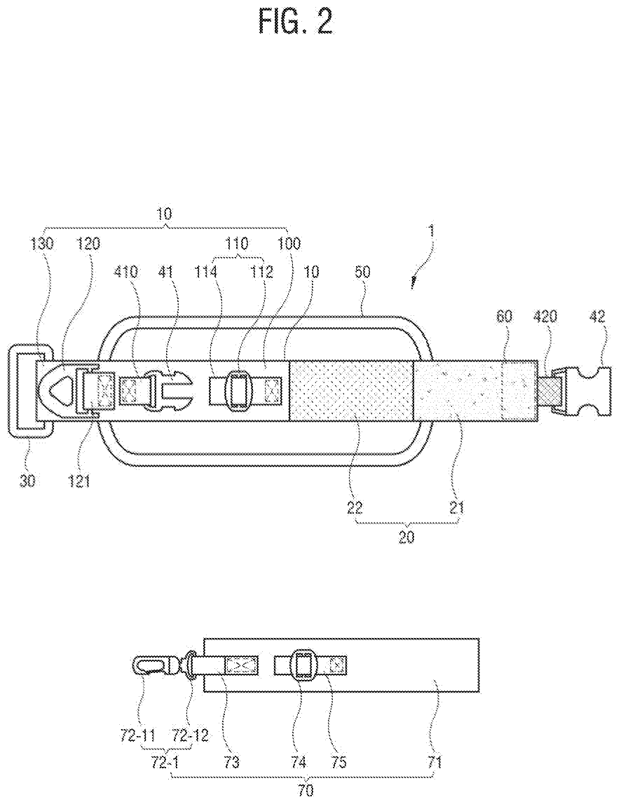

FIG. 1 illustrates an example that a pressure instruments 1 according an embodiment of the disclosure is worn on a wrist of a patient, and FIG. 2 illustrates that the pressure instruments 1 is opened. The pressure instruments 1 is dually fastened and locked onto a wrist of a patient as a part of a belt unit 10 with a first coupler 21 and a second coupler 22 bonded to each other is curved toward an outer surface after passing through a switch ring 30, and a first fastener 41 coupled to the outer surface in a second end region of the belt unit 10 by a first fastening support band 410 is engaged with and locked to a second fastener 42 coupled to a first end region by a second fastening support band 420.

The pressure instruments 1 includes the belt unit 10, a coupler 20, the switch ring 30, a fastener 40, a padding portion 50, an index pocket 60, and a holding belt 70.

The belt unit 10 is shaped like a long strap and includes the inner contact surface to be in contact with and wound around a wrist of a patient. The belt unit 10 includes a belt main body 100, a length adjuster 110, a belt ring 120, and a switch ring support band 130. The belt main body 100 has a strap shape. On the outer surface of the belt main body 100, the length adjuster 110 is mounted to adjust the length of the belt main body 100. The length adjuster 110 includes a belt length adjustment ring 112 and a belt length adjustment band 114. The belt ring 120 is supported in a belt ring support band 121 fastened and mounted to the outer surface of the belt main body 100. The belt length adjustment ring 112 is shaped like two adjacent quadrangular rings. The belt length adjustment band 114 is locked after passing through the two adjacent quadrangular rings of the belt length adjustment ring 112. The belt length adjustment band 114 is made of a somewhat stiff textile to keep its shape after passing through the two adjacent quadrangular rings of the belt length adjustment ring 112. Thus, the length of the belt main body 100 is adjusted to fit the thickness of a patient's wrist.

The switch ring support band 130 holds and supports the switch ring 30.

The coupler 20 includes the first coupler 21 and the second coupler 22 which are mounted in sequence from the first end region of the belt main body 100 to a predetermined length section of the outer surface and attached to each other. The coupler 20 may include a Velcro supporting band that can be attached in the lengthwise direction of the belt main body 100, and the first coupler 21 and the second coupler 22 are attached with the Velcro supporting band in the lengthwise direction of the belt main body 100. The first coupler 21 and the second coupler 22 may form a pair of Velcro straps which stick together when they come into contact with each other.

The switch ring 30 is rotatably mounted in the lengthwise direction of the belt main body 100. The switch ring 30 is shaped like a ring large enough to allow the belt main body 100 to pass therethrough, and is rotatably supported in the lengthwise direction of the surface of the belt main body 100. The switch ring 30 is fastened to the second end region of the belt main body 100 and allows the first coupler 21 and the second coupler 22 to pass therethrough and be bent in the first end region of the belt main body 100 so that the first coupler 21 and the second coupler 22 can stick together.

In the fastener 40, when the first end portion of the belt main body 100 passes through the switch ring 30 and compresses a patient's wrist, the belt main body 100 is bent toward the outer surface so that the first coupler 21 and the second coupler 22 can contact and stick together, and the first fastener 41 and the second fastener 42 are positioned in a region overlapping with the regions of the first coupler 21 and the second coupler 22 coupled on the surface of the belt main body 100 so that the first fastener 41 and the second fastener 42 can be fitted to each other, thereby achieving a dual lock. The first fastener 41 and the second fastener 42 are supported on the belt main body 100 by the first fastening support band 410 and the second fastening support band 420, respectively.

The fastener 40 includes a pair of locking units pressed or released at opposite sides along the widthwise direction of the belt main body 100. The first fastener 41 may be shaped like a hook, but not limited to this shape. The second fastener 42 may be shaped to accommodate and hold the hook, but not limited thereto. The fastener 40 may be released from the dual lock by using at least two fingers for pressing one pair of locking units in the widthwise direction of the belt main body 100, thereby preventing the dual lock from being released even though a patient presses the back of his/her hand against a wall or other protruding objects. The fastener 40 is allowed as long as the first fastener 41 and the second fastener 42 are fitted to each other, but prevented from being released when it is pressed toward the outer surface of the belt main body 100.

The fastener 40 is fastened to the belt main body 100 by the first fastening support band 410 and the second fastening support band 420, in which at least one of the first fastening support band 410 and the second fastening support band 420 is elastically stretchable. If a patient has a thick wrist but neither the first fastening support band 410 nor the second fastening support band 420 is stretchable, the wrist of the patient may be excessively pressed to thereby cause a problem in blood circulation. Therefore, it is preferable that at least one of the first fastening support band 410 and the second fastening support band 420 is stretchable.

The padding portion 50 is attached to the inner contact surface of the belt main body 100. The padding portion 50 is made of a soft material to protect a wrist from the compression of the belt main body 100.

The index pocket 60 is made of a transparent material and attached to the inner contact surface of the belt main body 100 in the region of the first coupler 21. In the index pocket 60, a patient's name, information and the like are provided so that the patient can be identified.

The holding belt 70 includes a holding belt main body 71, an end holder 72, a swivel support band 73, a holding belt adjustment ring 74, and a holding belt adjustment band 75. The holding belt main body 71 is shaped like a long strap. The end holder 72 includes a first end holder 72-1 and a second end holder 72-2. The first end holder 72-1 includes a hooking swivel 72-11 and a swivel support ring 72-12. The hooking swivel 72-11 is connected to the belt ring 120, and the swivel support ring 72-12 rotatably connected to the hooking swivel 72-11 is supported in the holding belt main body 71 by the swivel support band 73.

The swivel support ring 72-12 is shaped to have a protruding pillar and an extended portion transversely extended from the end of the protruding pillar. At one side of the hooking swivel 72-11, a hook shape is provided to accommodate the protruding pillar of the swivel support ring 72-12 and hooked to the extended portion. Further, the hooking swivel 72-11 may be hooked to and unhooked from the belt ring 120 by pressing a part of a circular ring to be opened for connection and disconnection. To easily evacuate a patient in a fire or the like situation, the hooking swivel 72-11 needs to be unhookable.

The second end holder 72-2 is formed as the holding belt main body 71 forms a ring by the holding belt adjustment ring 74 and the holding belt adjustment band 75 to be described later. The second end holder 72-2 may be held in and fastened to a stationary structure. The holding belt 70 includes the holding belt adjustment ring 74 and the holding belt adjustment band 75, which have the same function as the length adjuster 110, in the lengthwise direction in order to adjust the length of the holding belt 70.

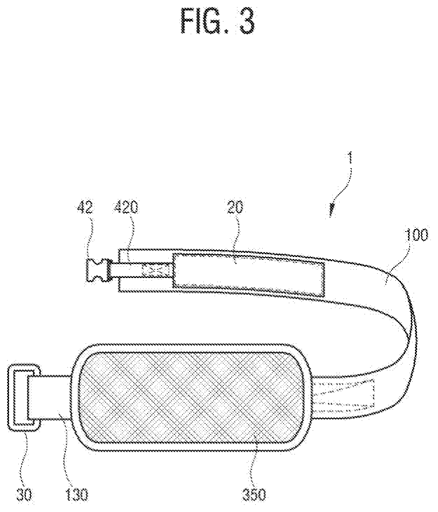

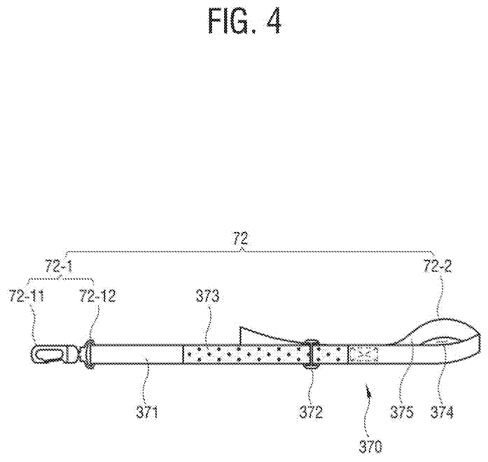

FIG. 3 illustrates a padding portion 350 and a holding belt 370 according to another embodiment of the disclosure. FIG. 4 illustrates a holding belt 370 according to another embodiment of the disclosure.

The padding portion 350 is attached to the inner contact surface of the belt main body 100, and the inner contact surface of the padding portion 350 may have an airy structure as shown in FIG. 3. The inside of the padding portion 350 is made of an elastic material so that a patient can feel comfortable. Further, mesh fabric may be used so that the padding portion 350 can be well ventilated. However, without limitations, the padding portion 350 may be made of a thick elastic fiber having an airy structure. Although it is not illustrated in FIG. 3, the first coupler and the second coupler of the coupler 20 may be successively attached to the outer surface of the belt main body 100. However, the first coupler and the second coupler may not be successively connected, but separated at a predetermined distance.

In FIG. 4, unlike the holding belt 370 of FIG. 2, the holding belt 370 may be shaped as if the length adjuster 110 is enlarged, and include rubber projections 373 on the surface thereof to contact and hold a holding belt adjustment ring 372. Further, the holding belt 370 may be tied to a pillar or the like structure by passing the first end holder 72-1 through a first end region of a through portion sewed to have a double-layered belt structure at the end of the second end holder 72-2. The end holder 72 may employ only one of the first end holder 72-1 and the second end holder 72-2, or combination of them. A holding belt main body 371 may be made of at least one inelastic fabric or elastic fabric. Like a general belt, the holding belt main body 371 may be provided with a prong and a prong hold to adjust its length, but not limited thereto. However, the holding belt main body 371 may have any structure as long as it can be properly changed in length and then locked.

Here, the second end holder 72-2 is formed as the holding belt main body 371 forms a belt through-hole 374 shaped like a ring by the holding belt adjustment ring 372 and the holding belt adjustment band 375. When a user makes the first end holder 72-1 be moved across a stationary structure, i.e. a lower bed frame and then passed through the belt through-hole 374 that does not cross the lower bed frame, the holding belt main body 371 is fastened to and held in the lower bed frame. In this manner, the holding belt 370 can hold the pressure instruments 1.

The pressure instruments 1 may be provided in a glove form.

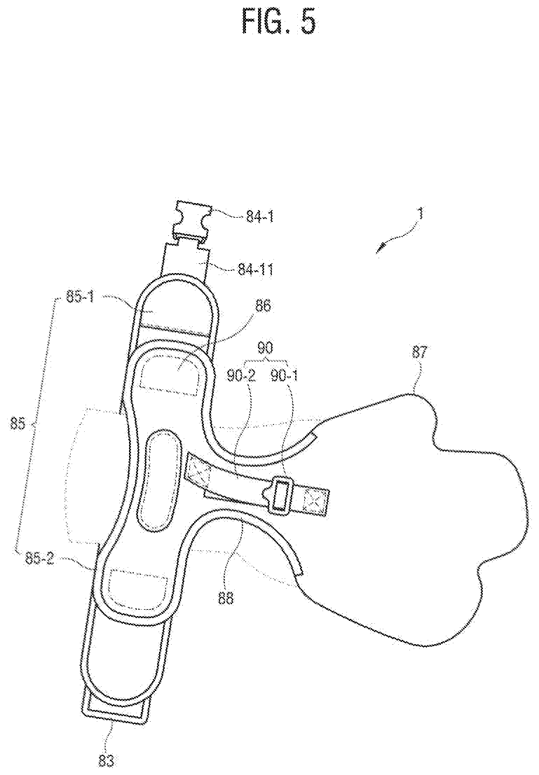

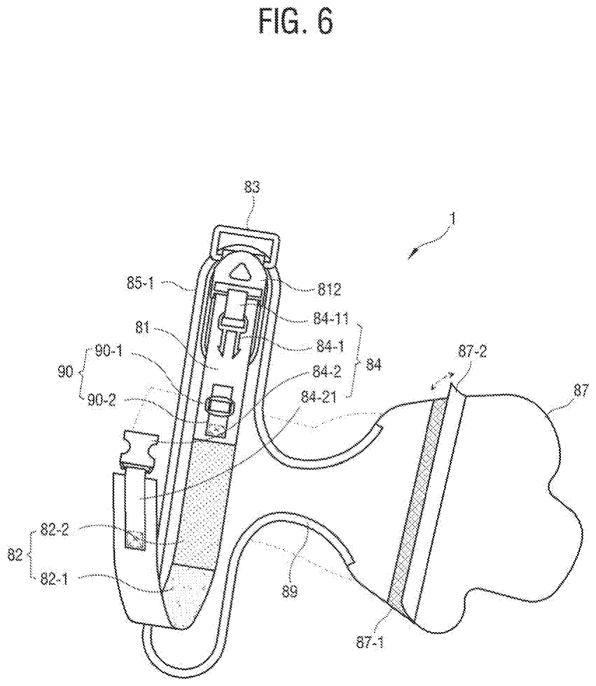

FIG. 5 is a perspective view of an upper connector 88 in the pressure instruments 1. FIG. 6 is a perspective view of a lower connector 89 in the pressure instruments 1.

In addition to the foregoing configuration, the glove-type pressure instruments 1 may include a belt main body 81, a hook buckle 84, a finger accommodator 87, and a length adjuster 90. A belt main body 81 includes a support ring 812 and a padding portion 85.

A padding portion 85 of a belt main body 81 connects with the finger accommodator 87 for accommodating a patient's fingers by the upper connector 88 and the lower connector 89. When the pressure instruments 1 is worn on a hand of a patient, the upper connector 88 connects the finger accommodator 87 and the padding portion 85 of the belt main body 81 along the upper portion, i.e. the back of his/her hand. The lower connector 89 connects the finger accommodator 87 and the padding portion 85 of the belt main body 81 along the lower portion, i.e. the palm when worn on the hand of the patient. The finger accommodator 87 is similar to a partial shape of a mitten, but a portion surrounded by the upper connector 88 and the lower connector 89 does not fully cover the hand of the patent so that the hand of the patient can be exposed to the outside unlike the mitten. Thus, the fingers of the patient accommodated in the finger accommodator 87 are ventilated unlike those in the mitten.

The inner contact surface of the belt main body 81 may be provided with an index pocket 86 made of a transparent material. In the index pocket 86, a patient's name, information and the like are provided so that the patient can be easily identified.

The support ring 812 provided in the belt main body 81 connects with a hooking swivel 72 of the support belt 70 shown in FIG. 1. The support belt 70 is connected to a stationary support bar such as a bed pillar, a chair, etc. of a patient, and supports the belt main body 81 dually fastened and locked to the patient's wrist. The support belt 70 needs to be adjusted in length according to a patient's postures, and therefore a belt adjustment ring 75 and a holding belt adjustment band 76 are provided on the outer surface of the support belt 70 to adjust the length.

It is shown that an inner contact surface of a first padding portion 85-1 is connected to the belt main body 81. The inner contact surface of the first padding portion 85-1 connects with the second padding portion 85-2 connected to the finger accommodator 87 and the upper connector 88. The inside of the second padding portion 85-2 is made of an elastic material so that a patient can feel comfortable like the padding portion 50. Ventilation may be achieved in a portion formed by the upper connector 88 and the lower connector 89 connected to the belt main body 81, but mesh fabric for the ventilation may be applied to the finger accommodator 87. The finger accommodator 87, the upper connector 88 and the second padding portion 85-2 are formed as a single body. However, the finger accommodator 87 and the second padding portion 85-2 are separately provided and stick together by Velcro at first and second sides.

When it is hot, a patient's hand may be perspiring, thereby causing skin troubles. To prevent this, the upper connector 88 may be shaped to partially expose a hand of a patient to the outside. Thus, a patient's fingers accommodated in the finger accommodator 87 are smoothly ventilated. The length adjuster 90 may be provided in an outer surface region of the upper connector 88. Thus, it is possible to adjust the length of the second padding portion 85-2 connected to the padding portion 85 of the belt main body 81 and the finger accommodator 87 in order to prevent a patient's hand from freely moving and getting out of the finger accommodator 87. For example, the length of the length adjuster 90 may be decreased for a patient who has a small hand.

The pressure instruments 1 of FIG. 6 includes the belt main body 81 shaped like a long strap and having an inner contact surface to be in contact with and wound around a wrist. The belt main body 81 is mounted with a Velcro unit 82 including a first Velcro portion 82-1 and a second Velcro portion 82-2 which are mounted in sequence from the first end region of the belt main body 81 to a predetermined length section of the outer surface and attachable to each other.

The Velcro unit 82 may include a Velcro supporting band attachable in the lengthwise direction of the belt main body 81, and the first Velcro portion 82-1 and the second Velcro portion 82-2 may be attached to the Velcro supporting band in the lengthwise direction of the belt main body 81.

The first Velcro portion 82-1 and the second Velcro portion 82-2 include small fiber hooks and small fiber loops in which the small fiber hooks engage, and stick together when they are in contact with each other.

In the belt main body 81, a switch ring 83, which is fastened to the second end region of the belt main body 81 and allows the first Velcro portion 82-1 and the second Velcro portion 82-2 to pass therethrough and bend in the first end region so that the first Velcro portion 82-1 and the second Velcro portion 82-2 can contact and stick together, is rotatably mounted to the belt main body 81 in the lengthwise direction. The switch ring 83 is shaped like a quadrangular ring large enough to allow the belt main body 81 to pass therethrough, and is rotatably supported in the lengthwise direction of the surface of the belt main body 81.

The belt main body 81 is attached with a hook buckle 84 which includes a first buckle 84-1 fastened to the outer surface of the second end region of the belt main body 81, and a second buckle 84-2 fastened to the region of the Velcro portion 82-2 and engaged with the first buckle (84-1). When the first end portion of the belt main body 81 passes through the switch ring 83 until a wrist of a patient is compressed, the belt main body 81 is turned in the outer surface so that the first Velcro portion 82-1 and the second Velcro portion 82-2 can come into contact with each other and stick together. Like this, when the first Velcro portion 82-1 and the second Velcro portion 82-2 stick together, the first buckle 84-1 and the second buckle 84-2 are positioned in an overlapping region between the first Velcro portion 82-1 and the second Velcro portion 82-2 coupled to the belt main body 81 and then fitted to each other, thereby achieving the dual lock. Then, the hooking swivel 72 of the support belt 70 shown in FIG. 1 tied to the bed and the support ring 812 of the belt main body 81 are connected, so that the belt main body 81 in which a wrist of a patient is dually locked can be tied and supported, thereby restricting movement of the patient's hand and effectively preventing self-harm.

The hook buckle 84 include a pair of hooks pressed and released at lateral sides with respect to the widthwise direction of the belt main body 81. The first buckle 84-1 is shaped like a hook, but not limited thereto. The second buckle 84-2 is shaped to accommodate and hold the hook, but not limited thereto. The hook buckle 84 for the dual lock may be released by pressing the pair of hooks in the widthwise direction of the belt main body 81 with at least two fingers so that the dual lock can be prevented from being released even when a patient presses the back of his/her hand against a well or other protruding objects. The first buckle 84-1 and the second buckle 84-2 of the hook buckle 84 are fitted to each other, but may be coupled by various methods as long as the release is prevented even though they are pressed toward the outer surface of the belt main body 81.

The hook buckle 84 is fastened to the belt main body 81 by first and second fastening bands 84-11 and 84-21, and at least one of the first and second fastening bands 84-11 and 84-21 may be elastically stretchable. If a patient has a thick wrist but neither the first nor second fastening bands 84-11 nor 84-21 is stretchable, the wrist of the patient may be excessively pressed to thereby cause a problem in blood circulation. Therefore, it is preferable that at least one of the first and second fastening bands 84-11 and 84-21 is elastically stretchable. The belt main body 81 may be provided with the length adjuster 90 to fit the thickness of the patient. The length adjuster 90 may include a length adjustment ring 90-1 and a length adjustment belt 90-2. The length adjustment ring 90-1 may be shaped like two adjacent quadrangular rings. The length adjustment belt 90-2 passes through the two adjacent quadrangular rings of the length adjustment ring 90-1 and then locked. The length adjustment belt 90-1 is made of a somewhat stiff textile to keep its shape even after passing through the two adjacent quadrangular rings of the length adjustment ring 90-1. Thus, the length of the belt main body 81 is adjusted to fit the thickness of the patient's wrist.

The padding portion 85 is attached to the inner contact surface of the belt main body 81, and the inner contact surface of the padding portion 85 may have an airy structure. The inside of the padding portion 85 is made of a resilient material so that a patient can feel comfortable. Further, mesh fabric may be used so that the padding portion 85 can be well ventilated. However, without limitations, the padding portion 85 may be made of a thick elastic fiber having an airy structure.

Ventilation may be achieved by the upper connector 88 and the lower connector 89 connected to the belt main body 81. However, when it is hot, a patient's hand accommodated in the finger accommodator 87 may be perspiring, and therefore an airy structure, material, etc. may be used. For example, at least a part of the finger accommodator 87 may include a ventilation area 87-1 of which a structure, a material, etc. have higher ventilation effects than those of the other areas, and a ventilation cover 87-2 to open and close the at least the part, thereby opening the ventilation cover 87-2 at high temperature in summer but closing the ventilation cover 87-2 in winter. A patent who may harm himself/herself generally stays in a hospital room which is free from the outdoor temperature. Therefore, the pressure instruments 1 may be made of an airy material, or may be manufactured to have an airy structure.

A portion where the upper connector 88 and the lower connector 89 are connected to the belt main body 81 is always exposed to the outside as if it is cut. However, the ventilation cover 87-2 and the like cover may be provided to cover and protect a patient's hand when it is cold.

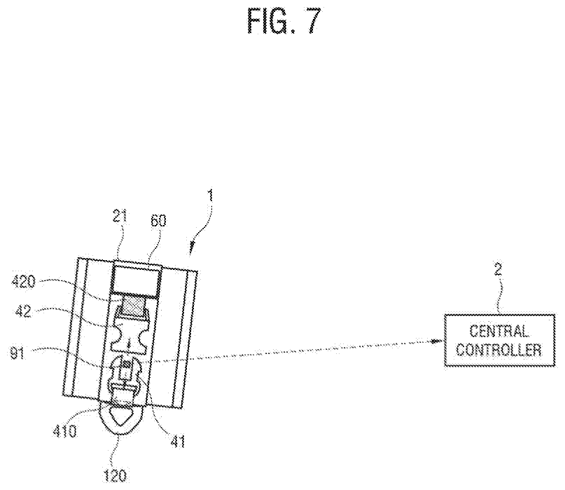

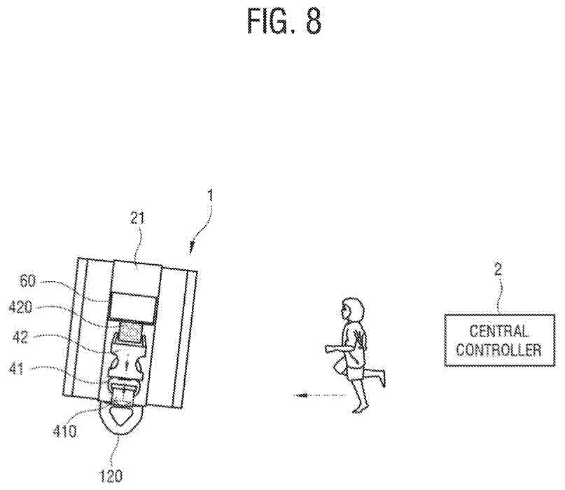

FIGS. 7 and 8 illustrate an alternative example of identifying whether the pressure instruments 1 is released. The pressure instruments 1 further includes a release sensor 91 provided at least one of between the first coupler 21 and the second coupler 22 and between the first fastener 41 and the second fastener 42 and detecting release. When a patient unties the pressure instruments 1, a primary lock between the first fastener 41 and the second fastener 42 and a secondary lock between the first coupler 21 and the second coupler 22 have to be released. Therefore, to prevent a patient from harming himself/herself, it is advantageous to detect the release of the lock between the first fastener 41 and the second fastener 42. The release sensor 91 may be provided between the first fastener 41 and the second fastener 42 rather than between the first coupler 21 and the second coupler 22. Preferably, the release sensor 91 may be provided between the first coupler 21 and the second coupler 22 as well as between the first fastener 41 and the second fastener 42.

In FIG. 7, when a patient or another person releases the lock between the first fastener 41 and the second fastener 42 of the pressure instruments, the release sensor 91 transmits a release signal to a central controller 2. A manager determines that the pressure instruments 1 is untied based on the release signal received in the central controller 2, and informs a guardian, a doctor, a nurse or a caregiver that the pressure instruments 1 is untied.

In FIG. 8, the guardian, the doctor, the nurse or the caregiver moves to the pressure instruments 1, and normally fastens the released first and second fasteners 41 and 42.

The release sensor 91 may be provided as magnetic sensors installed between the first coupler 21 and the second coupler 22 and between the first fastener 41 and the second fastener 42, and transmit the release signal to the central controller 2 when the first coupler 21 and the second coupler 22 or the first fastener 41 and the second fastener 42 are separated by more than a predetermined distance.

Besides the foregoing embodiment, other alternative embodiments will be described.

In the foregoing embodiment, the first coupler 21 and the second coupler 22 and the first fastener 41 and the second fastener 42 are used to compress and restrain a wrist of a patient, but the wrist of the patient may be restrained by an electromotive force. A fire signal may be detected or received from the outside. When the fire signal is detected or received, the pressure instruments 1 may be untied by the electromotive force. A locking unit may be provided to tie and untie the pressure instruments 1 by the electromotive force. The locking unit may be released from the locked state by the electromotive force. Further, the belt ring 120 may be shaped to be opened or closed.

With the foregoing pressure instruments 1, a patient is prevented from voluntarily releasing a locked state and tying capability is durably kept, thereby securely restraining a patient's hand and preventing the patient from harming himself/herself. The pressure instruments is stably maintained by stopping a patient from using fingers, and is clean and comfortable by improving ventilation. A manager is informed that a patient is untied, thereby having a further improved effect on preventing the patient from harming himself/herself.

1. A pressure instruments for preventing self-harm, comprising:
a belt unit shaped like a long strap and comprises an inner contact surface to be in contact with and wound around a wrist of a patient;
a coupler comprising a first coupler and a second coupler which are attached in sequence from a first end region of the belt unit to a predetermined length section of an outer surface and capable of being stick together; and
a fastener comprising a first fastener coupled to an outer surface of the second end region and a second fastener coupled to a region of the coupler and engaged with and locked to the first fastener.

2. The pressure instruments according to claim 1, wherein the fastener comprises a pair of locking units pressed and released.

3. The pressure instruments according to claim 1, wherein the fastener is fastened to the belt unit by a fastening band, and at least one of the fastening band is elastically stretchable.

4. The pressure instruments according to claim 1, further comprising:
a finger accommodator to accommodate fingers of the patient; and
an upper connector and a lower connector to connect the finger accommodator and the belt unit along an upper portion and a lower portion of a hand of the patient, respectively.

5. The pressure instruments according to claim 1, further comprising a holding belt connected to the belt unit and fastened and tied to a stationary structure.

6. The pressure instruments according to claim 5, wherein the holding belt comprises:
a holding belt main body shaped like a long strap;
a holding belt adjustment ring to adjust a length of the holding belt main body; and
an end holder supported in the holding belt main body and fastened and tied to the belt unit and the stationary structure.

7. The pressure instruments according to claim 6, wherein the holding belt main body comprises rubber projections throughout a predetermined section of an outer surface.

8. The pressure instruments according to claim 1, further comprising a release sensor provided at least one of between the first coupler and the second coupler and between the first fastener and the second fastener and detecting release.

9. The pressure instruments according to claim 1, further comprising a switch ring coupled to a second end region of the belt unit and allowing the first end region of the belt unit to pass therethrough and be bent reversely so that the first coupler and the second coupler can stick together.
